(19) United States
(12) Patent Application Publication
(10) Pub. No.: US 2014/0052265 A1
(43) Pub. Date: Feb. 20, 2014

(54) ROLLING-CONTACT KNEE PROSTHESIS

(71) Applicant: Massachusetts Institute of Technology, Cambridge, MA (US)

(72) Inventors: Alexander H. Slocum, JR., Huntington, WV (US); Daniel Goodman, Cambridge, MA (US); Michael Eilenberg, Cambridge, MA (US); Julia Titarelli, Ann Arbor, MI (US); Leslie Meyer, Humble, TX (US)

(73) Assignee: Massachusetts Institute of Technology, Cambridge, MA (US)

(21) Appl. No.: 13/968,224

(22) Filed: Aug. 15, 2013

Related U.S. Application Data
(60) Provisional application No. 61/683,415, filed on Aug. 15, 2012.
(60) Provisional application No. 61/866,171, filed on Aug. 15, 2013.

(51) Int. Cl. A61F 2/38

(52) U.S. Cl. CPC A61F 2/384
USPC 623/20.24

(57) ABSTRACT

A custom knee prosthetic includes a polycentric rolling contact joint, whose instantaneous center of rotation coincides with the instantaneous center of rotation of a patient's knee joint.

### CROSS REFERENCE TO RELATED APPLICATIONS

This application claims priority to U.S. Pat. App. 61/683,415, filed Aug. 15, 2012, the entirety of which is hereby incorporated by reference. This application also claims priority to U.S. Prov. Pat. App. 61/866,171, filed Aug. 15, 2013, the entirety of which is hereby incorporated by reference.

### STATEMENT OF GOVERNMENT INTEREST

This invention was made with government support under Contract No. W81XWH-09-2-0001 awarded by the U.S. Army Medical Research and Material Command. The government has certain rights in the invention.

### BACKGROUND

There remains a need for improved joint designs for knee joint prostheses.

### SUMMARY

A variety of embodiments are disclosed for knee joint prostheses, including joint replacements and knee braces. The prostheses may include, among other things, polycentric rolling contact joints. The rolling contact joints may be constructed such that the instantaneous center of rotation during knee flexure or extension coincides with the instantaneous center of rotation of the knee joint itself.

### BRIEF DESCRIPTION OF THE FIGURES

FIG. 1 is a schematic view of a four bar linkage model derived from a human knee.

FIG. 2 is a flowchart for creating a custom knee prosthesis for a patient.

FIG. 3 is a graph describing the geometry of joint components.

FIG. 4 is a side view of an exemplary rolling contact joint.

FIG. 5 is a side view of an exemplary geared rolling contact joint.

FIG. 6A is an exploded view of an exemplary rolling contact joint assembly.

FIG. 6B is an isometric view of an exemplary rolling contact joint assembly.

### DETAILED DESCRIPTION

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus the term “or” should generally be understood to mean “and/or” and so forth.

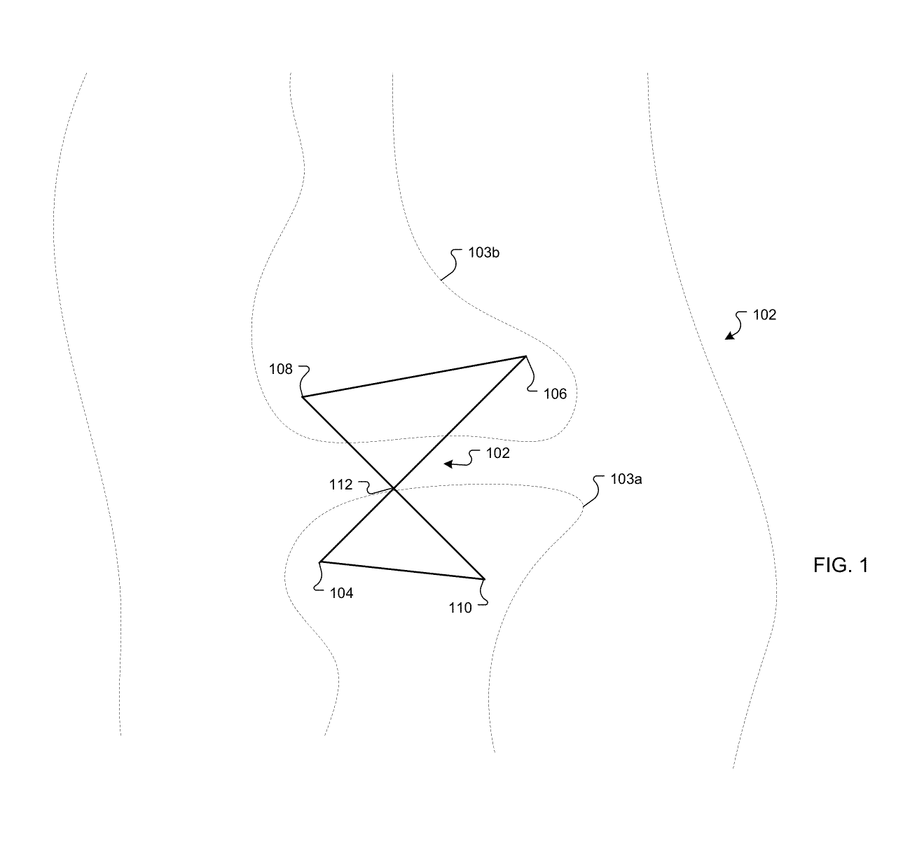

FIG. 1 is a schematic view of a four bar linkage derived from a human knee. The linkage 100 is shown superimposed against a schematic lateral radiograph of a knee 102 (shown in dashed lines), including a tibia 103a and a femur 103b to illustrate the anatomical context. The linkage 100 is assumed to lie in a sagittal plane, and is comprised of projections of four anatomical points onto the plane. The four anatomical points are the origin 104 and the insertion 106 of the anterior cruciate ligament (ACL), and the origin 108 and insertion 110 of the posterior cruciate ligament (PCL). Any specific sagittal plane on which to project the anatomical points may be chosen; for example, any sagittal plane passing between the midpoints of any pair of anatomical points noted above, or any location derived from a weighted average of the locations of any two or more of the anatomical points. Unless otherwise specified in this document, reference to these anatomical points should be understood to refer to their corresponding projections in the selected sagittal plane.

The four bar linkage includes links between each of these anatomical points, except: ACL origin 104 to PCL origin 108; and the ACL insertion 106 to the PCL insertion 106. For convenience in what follows, it will be assumed that the link from the ACL origin 104 to the PCL insertion 108 is fixed, and to the extent necessary, a coordinate system can be defined implementing this assumption. In some implementations, the link from the ACL origin 104 to the PCL insertion 108 may be considered as the ground link in the four-bar mechanism. The links from the ACL origin 104 to the ACL insertion 106 and the PCL origin 108 to the PCL insertion 110 may be referred to in this document as grounded links. However, any coordinate system in which any link is fixed (or no link is fixed) may be adopted without departing from the scope of the invention.

The human knee's natural motion is close to, but distinct from, the motion of a pin joint (in which one of the joint's components may be considered fixed, and the other rotates about a fixed center of rotation linking the components). More specifically, during a period of flexion or extension, the tibia 103a and femur 103b undergo a “polycentric” rotational motion. That is, at any instant during flexion or extension, there is an “instantaneous center of rotation” about which the femur and/or tibia are rotating, but the location of this instantaneous center of rotation changes as the knee continues to flex or extend due to the variable radii of the human femoral condyles. In the four bar linkage 100, this instantaneous center of rotation of the knee joint is approximated to a useful degree by the intersection point 112 of the links corresponding to the ACL and PCL (the grounded links).

It is advantageous for a knee prosthesis to mimic the natural kinematics of the knee, in particular accounting for a knee's polycentric rotation. Moreover, insofar as individuals' anthropometric variations are concerned, it is also advantageous for a particular prosthesis to mimic the specific kinematics of the patient for whom the prosthesis is intended. The techniques described herein are useful to produce such prosthetic devices or components thereof.

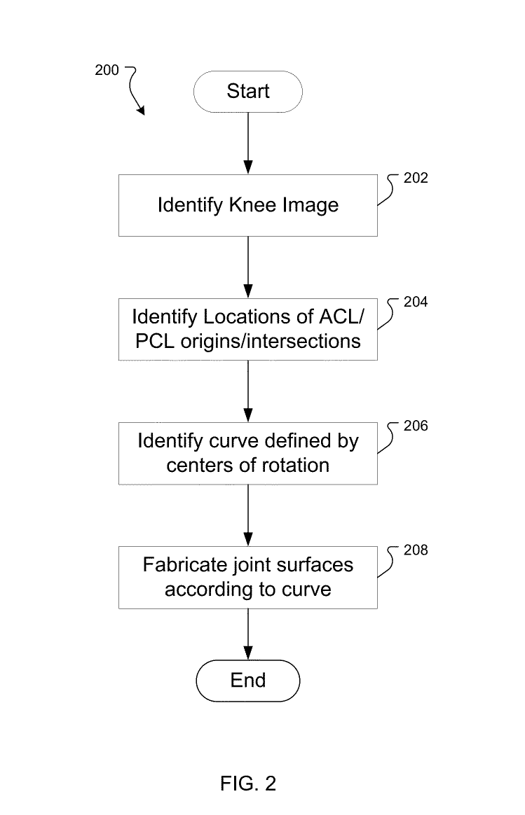

FIG. 2 is a flowchart for creating a custom knee prosthesis for a given patient. Advantageously, the method 200 can be used to produce a prosthesis (or components thereof) that accounts for the individual's unique anatomy as described above.

In step 202, an image of the patient's knee may be identified. In some implementations, the image may be a radiographic image such as an X-ray, magnetic resonance image (“MRI”), computed tomography (“CT”), or any other imaging modality sufficient to capture the requisite information about the ACL and PCL origins/insertions as described below. The image may be two dimensional or three dimensional. For two dimensional images, in some implementations, it is convenient to obtain the image in a sagittal plane so that positions of the anatomical points described above can be more easily located. For three dimensional images, the positions of the anatomical points described above can be projected onto a sagittal planar coordinate system.

In step 204, the locations of the origins and insertions of both the ACL and the PCL may be identified within a selected sagittal plane. These locations can be used to define a model four bar linkage as described above. In step 206, the trajectory of the instantaneous center of rotation (as the knee undergoes flexion or extension in a specified range of motion) are identified. In some implementations, the trajectory of the instantaneous center of rotation may be obtained by using the four bar linkage model described above. In some implementations, the instantaneous center of rotation may be obtained by other means.

The specified range of motion may include flexion from a tibio-femoral joint angle of 0 degrees (i.e., in which the knee is extended), to a joint angle of 175 degrees or more. In some implementations, it may be desirable to limit the range of motion (e.g., from 0 to 135 degrees) for the safety of the patient or for other reasons.

The instantaneous centers of rotation identified in step 206 collectively comprise a curve. However, the curve may appear differently when expressed in different coordinate systems or when different patients' anatomy is used to construct the four-bar linkage.

In some implementations, the solution of the forward kinematics problem in different coordinate systems may be used to design different components of a knee prosthesis. For example, a coordinate system in which the tibia is fixed (as described above) can be used to describe a surface of a joint component that can be advantageously coupled to the tibia. Similarly, a coordinate system in which the femur is fixed can be used to described a surface of a compatible joint component that can advantageously be coupled to the femur. For joints having mating surfaces constructed this way, the point of rolling contact between the joint components is, to a useful degree, coincident with the instantaneous center of rotation of the physical knee joint during flexure and extension.

However, the technique described in the previous paragraph is not the only way to arrive at joint component geometry. In particular, the joint components that result from the previous paragraph are necessarily of complementary convexity (i.e., one joint surface is convex, and it mates with a concave joint surface). By contrast, joints having two mating convex surfaces are possible, which still possess the property that the point of rolling contact between the joint surfaces is coincident to a useful degree with the instantaneous center of rotation of the physical knee joint.

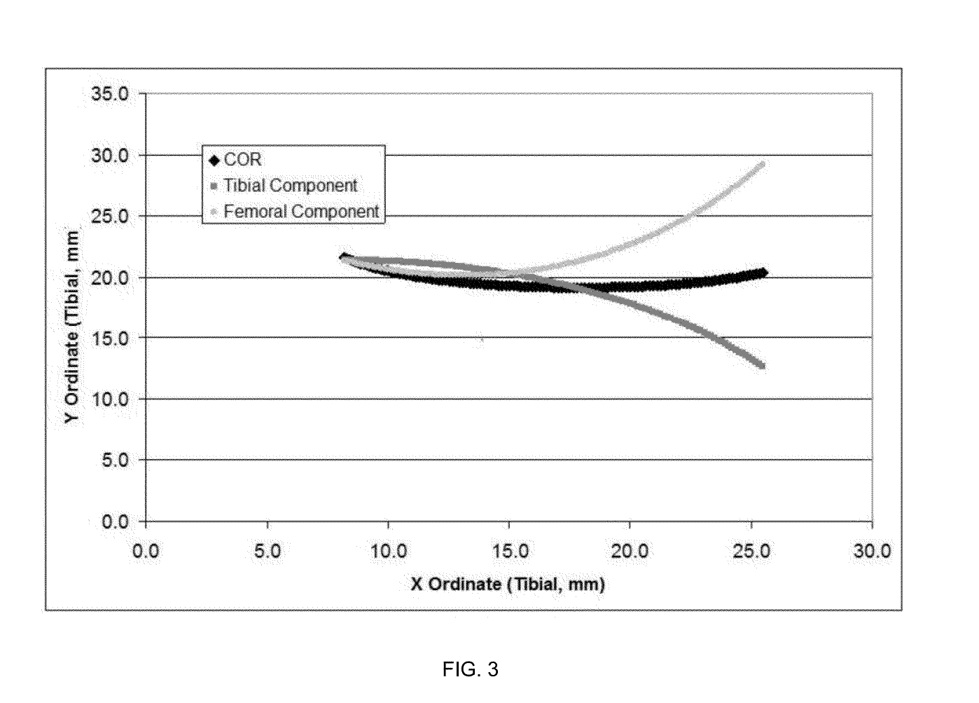

Referring to FIG. 3, one way to construct such “convex/convex” joints is illustrated. FIG. 3 is a plot of a typical curve 302 of the instantaneous centers of rotation (“COR”). The coordinate system of FIG. 3 is such that origin is coincident with the insertion of the ACL on the anterior tibia, the Y-axis is orthogonal to the X-axis, which is parallel to the horizontal, and the coordinate system lies in the relevant sagittal plane. An arbitrary reference curve 304 (e.g., a convex curve) may be defined to describe one of the joint's surface's geometry. The reference curve 304 may be coincident with at least one point of the COR curve 302. Then, a difference between the COR curve 302 and the reference curve 304 can be computed, yielding a difference curve (not shown). By adding a constant term, the difference curve can be shifted so that it is coincident with both the COR curve 302 and the reference curve 304. The shifted curve 306 describes the shape of a surface that mates with the surface described by the reference curve 304. For a typical COR curve 302, a convex reference curve 304 may yield a convex curve 306. In some implementations, the reference curve 304 may be a circular arc.

Techniques for designing joint surface geometries are described further in Rolling Contact Orthopaedic Joint Design by Alexander Henry Slocum Jr. (PhD Thesis, Mechanical Engineering Department, Massachusetts Institute of Technology, 2013), the entirety of which is incorporated by references herein.

Referring back to FIG. 2, in step 208, joints having components with appropriate mating surfaces are constructed, such that the point of contact as the interface of mated components coincides with the instantaneous center of rotation of the knee joint. Appropriate materials for joint construction are describe in more detail below.

Joints fabricated according to method 200 also possess the property that the surfaces roll (as opposed to slip) relative to each other during articulation. In practice, small design or manufacturing deviations from the above description may introduce a small degree of slippage during joint articulation. In this document, a degree of slippage during articulation that does not result in additional discomfort or adverse medical consequences (vs. no slippage) to a patient during the anticipated lifetime of the fabricated joint is regarded as insubstantial.

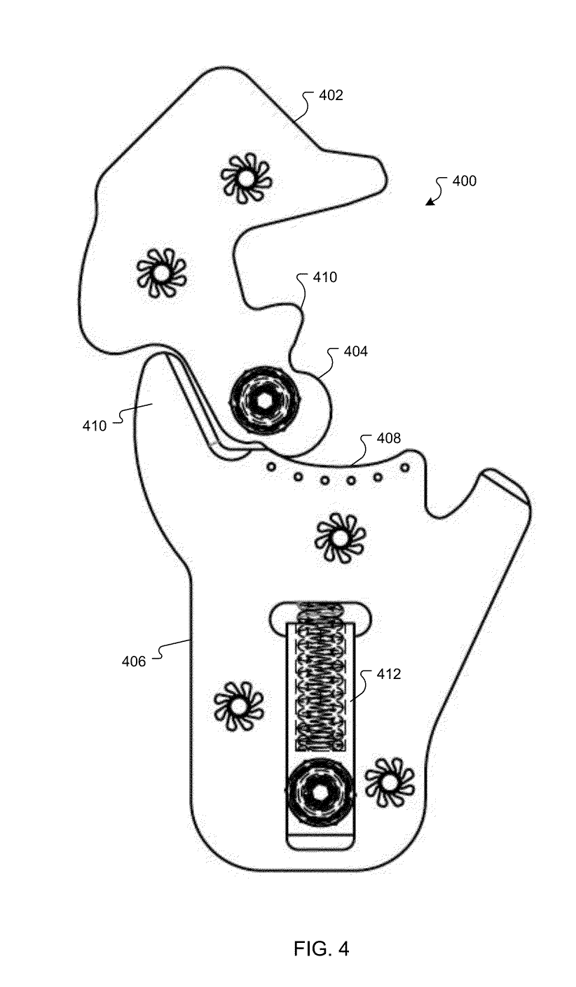

FIG. 4 is a side view of an exemplary rolling contact joint. The joint 400 may include a first component 402 having a first surface 404 and a second component having a second surface 406. In this exemplary joint 400, the surfaces 404 and 406 have been constructed using the technique of expressing the curve of instantaneous centers of rotation in coordinates in which the femur (for surface 404) and the tibia (for surface 406) remain fixed. As described above, during articulation of the joint 400, the first component 402 rolls across the second component 406 at a point of contact between the surfaces 404 and 408.

Each component 402, 406 may include a protrusion 410 operable to limit the joint's range of motion in one or both directions. In some implementations, the protrusions 410 are positioned to permit a range of motion corresponding to a tibio-femoral angle range of a typical healthy knee (i.e., between 0 degrees and 175 degrees). In some implementations, the protrusions 410 may be positioned to permit a range of motion corresponding to some other range of tibio-femoral angle; e.g., 0 to 135 degrees. A lesser angle range can be advantageous to mitigate the risk of certain injuries with certain patients.

Although the protrusion 410 is shown as structure that physically interferes with the motion of the joint 400, other implementations are possible. For example, corresponding structures may be employed to mechanically resist (or entirely limit) motion beyond a pre-defined range. More particularly, magnetic or electromagnetic structures, hydraulics, actuators, springs, or the like may be used to provide a resistive or limiting reactionary force to motion beyond a pre-defined threshold.

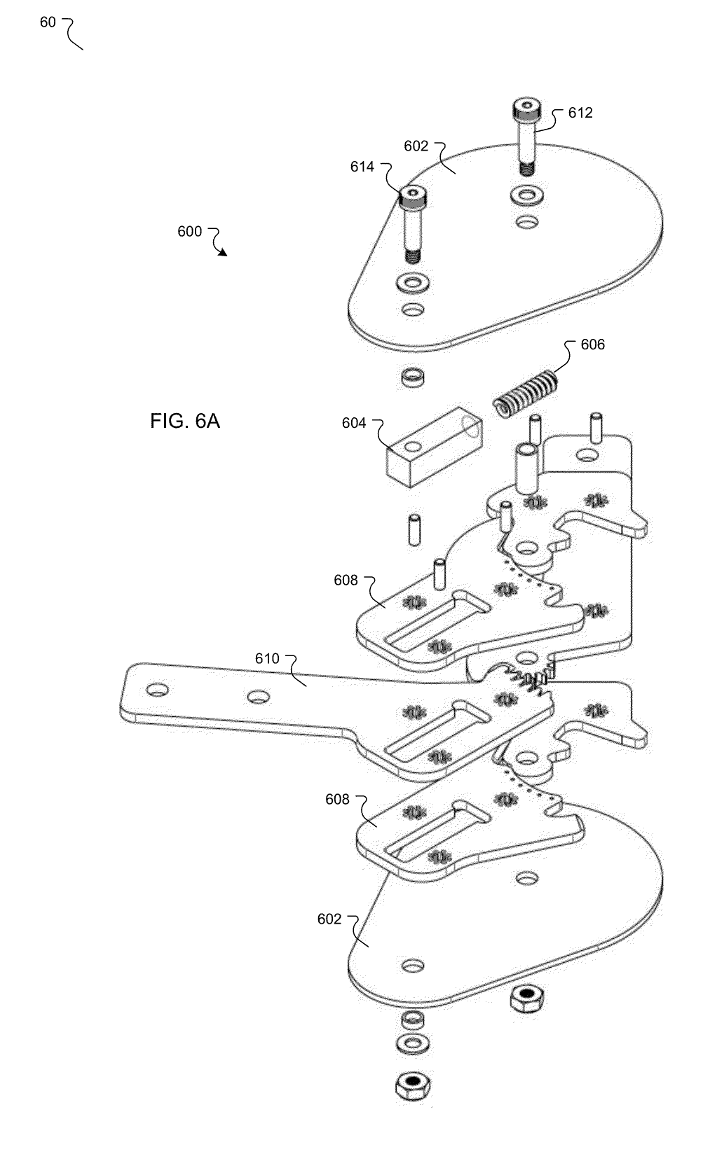

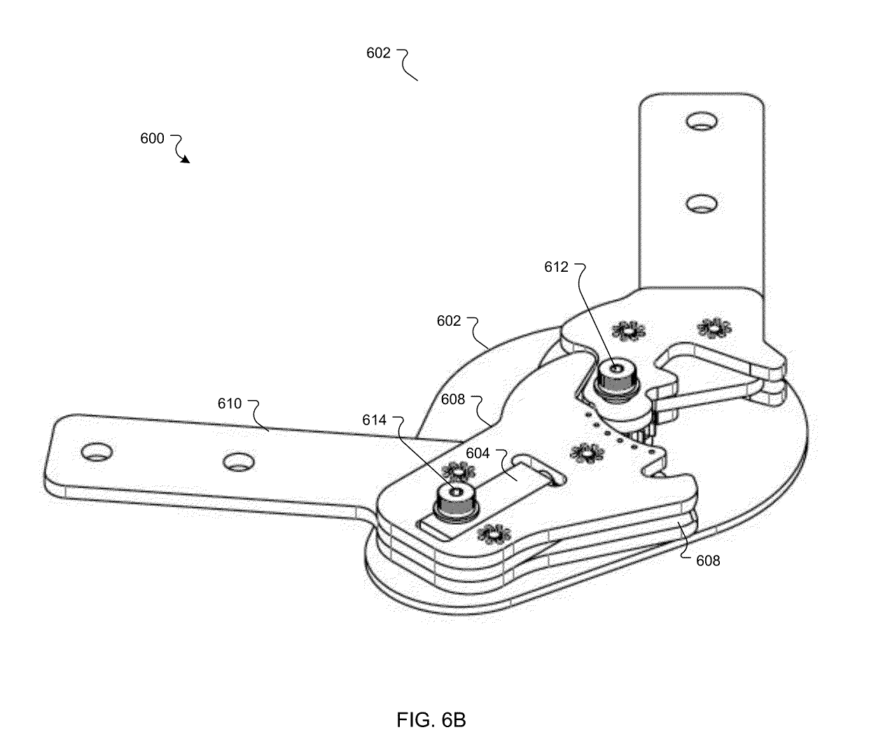

The joint 400 may include a pre-load spring 412. The spring 412 may be operable to bias the component second component 406 towards the first component 402, thereby mitigating the risk of undesirable separation during use. In order to effectively provide the pre-load, one end of the spring 412 may be mechanically coupled to the second component 406, while another end of the spring 412 may be coupled to a different component. (See FIGS. 6A and 6B for exemplary embodiments.)

In some implementations, the pre-load force provided by the spring 412 may be great enough to maintain engagement of the components 402, 406 under worst case conditions with respect to the risk of the components being separated (e.g., motions akin to deep knee squats). In some implementations, the biasing force may be between 40N and 60N. In some implementations, the biasing force may be determined for the particular patient based on the patient's individual requirements.

Although a spring 412 is shown in the exemplary FIG. 4, in general any structure operable to bias one component 402, 406 into the other may be employed. Such structures include, but are not limited to: magnets, tension bands or straps, linkages, or any such manner of constraining two cam surfaces relative to one another.

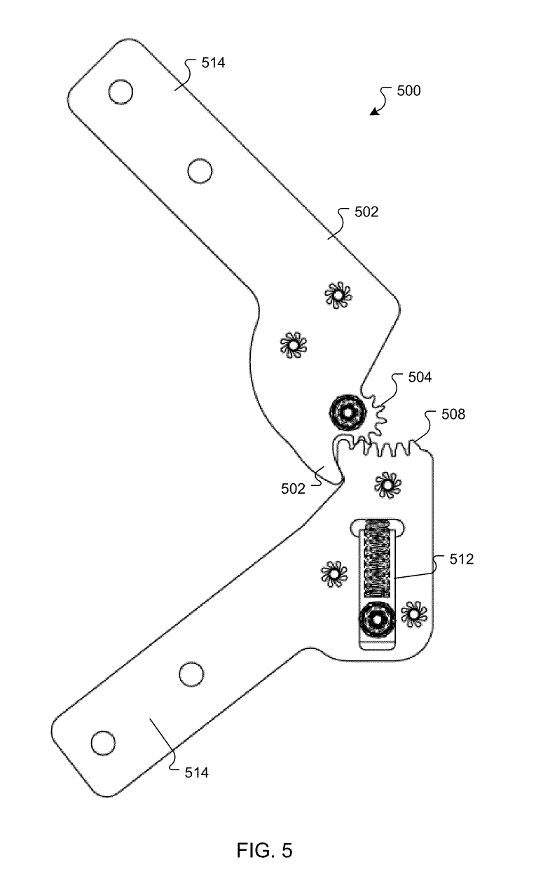

FIG. 5 is a side view of an exemplary geared joint. The geared joint 500 includes a first component 502 having a first geared surface and a second component 504 having a second geared surface 508. The surfaces 504, 508 can be defined as in the previous rolling contact joint example, except for the presence of mating trapezoidal gear teeth. Similarly to the previous example, the components 502, 506 may each include a protrusion 510 to limit flexure within a desired range, and the geared joint 500 may include a pre-load spring 512.

The geared joint 500 may also include additional couplings 514 extending away from the gears. These couplings may be useful for attaching the geared joint 500 to other structures. For example, in embodiments in which a geared joint 500 is included in a knee brace, the additional couplings 514 are useful to attach to cuffs or similar structures that are operable to removably couple the knee brace to a patient's tibia or femur. In embodiments in each a geared joint 500 is included in an in vivo application such as a knee replacement, the additional couplings 514 can be used to irremovably couple the components 502, 506 to a patient's femur or tibia.

Although FIG. 4 and FIG. 5 illustrated a rolling contact joint and a geared joint respectively, it may be understood that hybrids of these exemplary structures may be used. For example, a hybrid joint may have components with “partially geared” surfaces that articulate compatibly with the natural kinematics of a patient's knee joint.

FIG. 6A is an exploded view of an exemplary rolling contact joint assembly. FIG. 6B is a non-exploded view of the same assembly, except for a face plate that has been suppressed in order to reveal the remaining components. The face plate may be employed in order to mitigate parasitic torques on the joint's articulating surfaces.

The assembly 600 may include two face plates 602 (only one of which is shown in FIG. 6B), a pre-load spring case 604, a pre-load spring 606, two rolling contact joints 608, and a geared joint 610. The various joints 608-610 may include custom surfaces (or geared surfaces) that each, as described above, articulate in a way that matches the kinematics of a patient's knee. Although two rolling contact joints 608 and a single geared joint 610 are shown in FIGS. 6A and 6B, in principle any number of either component (or hybrids thereof) can be used.

Using a combination of a rolling contact joint 608 and a geared joint 610 has certain advantages over using only one type of either joint. For example, a geared joint 610 has the advantage that it is relatively resistant (compared to a rolling contact joint only) to parasitic torques. However, a rolling contact joint is relatively resistant to increased loads, e.g. from the weight of the patient. Thus, in combination, one or more rolling contact joints can advantageously help bear a patient's load (thereby prolonging the expected life of the geared joints), while one or more geared joints can advantageously mitigate undesirable torque, thereby prolonging the expected life of the rolling contact joints and the constraint mechanisms.

The face plates 602 may provide various advantages. For example, the face plates may shield the moving surfaces of the various joints 608, 610 in the mechanism 600, thereby mitigating the risk of damage to the mechanism or injury to the patient or third party. Moreover, one or both face plates 602 may advantageously serve as an anchor for certain components of the mechanism and help to mitigate parasitic torques. For example, the bolt 612 couples one component of each joint 608, 610 to the face plates 602, while the other component of each joint 608, 610 can “float” relative to the face plates 602. At the same time, the pre-load spring contacts the floating components of each of the joints 608, 610, thereby helping to ensure adequate engagement of the relevant components of each joint 608, 610.

In various embodiments, the structures described in FIGS. 3-6A, 6B can be constructed from any suitable material or combination of materials. In some implementations, the materials can include metal (e.g., stainless steel, aluminum, titanium, etc.). The materials can also include non-metals such as ceramics, plastics, or still other materials. The particular choice of material from which to fabricate a component is influenced by conventional design considerations, such as the intended component lifetime, cost sensitivity, operating environment (e.g., propensity to corrode certain metals in external applications; risk of causing an adverse reaction in a patient for in vivo applications, etc.), load capacity and other required mechanical tolerances, and the like.

The meanings of method steps of the invention(s) described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

Thus for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X′s participation in causing the step to be performed.

While particular embodiments of the present invention have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A device comprising:
a first component having a first surface; and
a second component having a second surface; wherein:
the first component is configured to:
couple to a human leg below a knee;
maintain rolling contact with substantially no slippage between the first surface and the second surface at a point of rolling contact during a period of flexion or extension of the knee; and
at each moment of flexion or extension, rotate with respect to an instantaneous center of rotation of the knee; and wherein the second component is configured to:
couple to the human leg above the knee;
maintain rolling contact with substantially no slippage between the first surface and the second surface at the point of rolling contact during the period of flexion or extension of the knee; and
at each moment of flexion or extension, rotate with respect to the instantaneous center of rotation of the knee; and wherein
at each moment of flexion or extension, the point of rolling contact between the first surface and second surface is coincident with the instantaneous center of rotation of the knee.

2. The device of claim 1, in which the first and second components are each configured to removably couple to the human leg.

3. The device of claim 1, in which the first component is configured to irremovably couple to a first bone in the human leg and the second component is configured to irremovably couple to a second bone in the human leg.

4. The device of claim 1, wherein the period of flexion or extension of the knee can accommodate any tibio-femoral angle between −10 degrees and 175 degrees.

5. The device of clam 1, further comprising a third component configured to provide a force biasing the first component to remain in contact with the second component.

6. The device of claim 5, wherein the third component includes a spring loaded coupling between the first component and the second component.

7. The device of claim 1, in which the first surface and second surface each include a plurality of gear teeth wherein the gear teeth are positioned to mate as the first component rolls with respect to the second component.

8. The device of claim 1, further comprising a third component and a fourth component, wherein:
the third component:
is configured to rigidly couple to the human leg above the knee; and
includes a third surface having gear teeth; and
the fourth component:
is configured to rigidly couple to the human leg below the knee; and
includes a fourth surface having gear teeth; and
the gear teeth of the third and fourth surfaces are configured to mate throughout the period of flexion or extension.

9. The device of claim 8, in which the third component is coupled to the first component, and the fourth component is coupled to the second component.

10. The device of claim 1, in which a first curve defined by the instantaneous centers of rotation of the first component with respect to the second component during the period of flexion or extension of the knee is equal to a second curve derived from a four bar linkage model of the knee, in which dimensions of the four bar linkage correspond to:
a first link between an origin and an insertion point of an anterior cruciate ligament (ACL);
a second link between an origin and an insertion point of a posterior cruciate ligament (PCL);
a third link between the insertion points of the ACL and PCL; and
a forth link between the origin points of the ACL and PCL; and
wherein the second curve is defined by the collection of intersection points of the first and second links of the four bar linkage as the knee.

11. The device of claim 1, in which a convexity of the first surface is complementary to a convexity of the second surface.

12. The device of claim 1, in which the first component includes protrusion that prevent flexion or extension beyond a pre-determined limit by interfering with the second component at the pre-determined limit.

13. The device of claim 1, in which the first surface and the second surface are convex, and in which a geometry of the second surface is given in a tibial coordinate system as a difference between:
a curve defined by the instantaneous centers of rotation of the knee joint during the period of flexion and extension and
a curve defined by the first surface plus a constant.

14. A method comprising:
from a radiographic image of a human knee, identifying locations in a sagittal plane of:
an insertion point of an anterior cruciate ligament (ACL);
an insertion point of a posterior cruciate ligament (PCL);
an origin point of the ACL; and
an origin point of the PCL;
based on the identified locations, identifying a trajectory of a point of intersection between a first line and a second line as the knee undergoes flexion or extension, in which the first line is defined by the insertion point of the ACL and the origin point of the PCL, and the second line is defined by the origin point of the ACL and the insertion point of the PCL;
constructing a rolling contact joint that includes a first component having a first surface and a second component having a second surface, wherein the first and second surfaces are dimensioned such that if the first surface rolls over the second surface, rolling point of contact between the first component and the second component traverses the trajectory of the point of intersection between the first line and a second line.

15. The method of claim 14, in which the first and second components are each configured to removably couple to a human leg containing the knee.

16. The method of claim 14, in which the first component is configured to irremovably couple to a first bone in the human leg and the second component is configured to irremovably couple to a second bone in the human leg.

17. The method of claim 14, wherein the period of flexion or extension of the knee can accommodate any tibio-femoral angle between 0 degrees and 175 degrees.

18. The method of claim 14, further comprising constructing a third component configured to provide a force biasing the first component to remain in contact with the second component.

19. The method of claim 18, wherein the third component includes a spring loaded coupling between the first component and the second component.

20. The method of claim 14, in which the first surface and second surface each include a plurality of gear teeth wherein the gear teeth are positioned to mate as the first component rolls with respect to the second component.

21. The method of claim 14, in which the knee is contained in a human leg, the method further comprising constructing a third component and a fourth component, wherein:
the third component:
is configured to rigidly couple to the human leg above the knee; and
includes a third surface having gear teeth; and
the fourth component:
is configured to rigidly couple to the to the human leg below the knee; and
includes a fourth surface having gear teeth; and
the gear teeth of the third and fourth surfaces are configured to mate throughout the period of flexion or extension.

22. The method of claim 21, further comprising coupling the first component to the third component, and coupling the second component to the fourth component.

23. The method of claim 14, in which a first curve defined by the instantaneous centers of rotation of the first component with respect to the second component during the period of flexion or extension of the knee is equal to a second curve derived from a four bar linkage model of the knee, in which dimensions of the four bar linkage correspond to:
a first link between an origin and an insertion point of an anterior cruciate ligament (ACL);
a second link between an origin and an insertion point of a posterior cruciate ligament (PCL);
a third link between the insertion points of the ACL and PCL; and
a forth link between the origin points of the ACL and PCL; and
wherein the second curve is defined by the collection of intersection points of the first and second links of the four bar linkage as the knee.

24. The method of claim 14, in which a convexity of the first surface is complementary to a convexity of the second surface.

25. The method of claim 14, in which the first component includes a protrusion that prevents flexion or extension beyond a pre-determined limit by interfering with the second component at the pre-determined limit.

26. The method of claim 14, in which the first surface and the second surface are convex, and in which a geometry of the second surface is given in a tibial coordinate system as a difference between:
a curve defined by the instantaneous centers of rotation of the knee joint during the period of flexion and extension and
a curve defined by the first surface
plus a constant.
